(19) United States
(12) Patent Application Publication
(10) Pub. No.: US 2026/0053418 A1
(43) Pub. Date: Feb. 26, 2026

(54) HEART SOUND SEGMENTATION METHOD, APPARATUS, ELECTRONIC DEVICE, AND READABLE STORAGE MEDIUM

(71) Applicants: SOUTHEAST UNIVERSITY, Nanjing (CN); GOERTEK INC., Weifang (CN)

(72) Inventors: Chengyu LIU, Weifang (CN); Yumin Li, Weifang (CN); Li Ling, Weifang (CN); Yaocheng Liu, Weifang (CN); Junjie Pan, Weifang (CN); Huan Li, Weifang (CN); Yanan Zhou, Weifang (CN); Chenxi Yang, Weifang (CN); Jianqing Li, Weifang (CN)

(21) Appl. No.: 19/375,546

(22) Filed: Oct. 31, 2025

Related U.S. Application Data
(63) Continuation of application No. PCT/CN2024/132614, filed on Nov. 18, 2024.

(30) Foreign Application Priority Data
Apr. 15, 2024 (CN) 202410454429.1

(51) Int. Cl. A61B 5/352; A61B 5/0205; A61B 5/024; A61B 7/04

(52) U.S. Cl. CPC A61B 5/352; A61B 5/0205; A61B 5/02416; A61B 7/04

(57) ABSTRACT

A heart sound segmentation method includes: synchronously collecting physiological signals, the physiological signals include heart sound signals; obtaining a segmentation threshold corresponding to the heart sound signal, and segmenting the heart sound signal based on the segmentation threshold to obtain a first heart sound and a second heart sound; and determining a systolic duration and a diastolic duration of the heart sound signal according to the first heart sound and the second heart sound.

### CROSS-REFERENCE TO RELATED APPLICATIONS

This application is a continuation application of International Application No. PCT/CN2024/132614, filed on Nov. 18, 2024, which claims priority to Chinese Patent Application No. 202410454429.1, filed on Apr. 15, 2024. All of the aforementioned applications are incorporated herein by reference in their entireties.

### TECHNICAL FIELD

The present application relates to the technical field of medical signal processing, and in particular relates to a heart sound segmentation method, an apparatus, an electronic device, and a readable storage medium.

### BACKGROUND

A key component of computer-assisted heart sound analysis is the segmentation of heart sound signals, specifically, distinguishing the exact locations of the first heart sound (S1), systole, second heart sound (S2), and diastole of each cardiac cycle. In each cardiac cycle, the first heart sound (S1) is caused by the closure of the mitral and tricuspid valves, the opening of the aortic and pulmonary valves, and the vibrations generated during ventricular contraction. The second heart sound (S2) is primarily caused by the closure of the aortic and pulmonary valves, coupled with vibrations caused by the reduction of blood flow in the aorta and pulmonary arteries. The systolic duration is the duration between S1 and S2, and diastolic duration is the duration from S2 to the start of the next cardiac cycle, S1. The accuracy of heart sound signal segmentation directly impacts the effectiveness of subsequent heart sound signal analysis.

In related arts, network models are usually used for heart sound segmentation. The segmentation algorithm based on the logistic regression hidden-semi Markov model (LR-HSMM) is one of the commonly used heart sound segmentation methods. However, the model-based heart sound segmentation method requires complex model calculations and has high computational complexity, which leads to high consumption of hardware and other resources. Therefore, there is an urgent need for a heart sound segmentation method for heart sound signals with low resource consumption.

The disclosure of the above background technology content is only used to assist in understanding the inventive concept and technical solution of the present application. It does not necessarily belong to the related art of this patent application, nor does it necessarily provide technical guidance. It is to provide general background information and does not necessarily constitute prior art.

### SUMMARY

The main purpose of the present application is to provide a heart sound segmentation method, an apparatus, an electronic device and a readable storage medium, aiming to solve the technical problem of how to reduce the resource consumption of the heart sound segmentation of heart sound signals on the server.

To achieve the above objectives, the present application provides a heart sound segmentation method, which includes:
synchronously collecting physiological signals, the physiological signals include heart sound signals;
obtaining a segmentation threshold corresponding to the heart sound signal, and segmenting the heart sound signal based on the segmentation threshold to obtain a first heart sound and a second heart sound; and
determining a systolic duration and a diastolic duration of the heart sound signal according to the first heart sound and the second heart sound.

In an embodiment, the physiological signal further includes an electrocardiogram signal and a pulse signal, and before the obtaining the segmentation threshold corresponding to the heart sound signal, the method further includes:
determining a QRS wave peak point of the electrocardiogram signal, and using a time corresponding to the QRS wave peak point as a first time;
determining a trough point of the pulse signal, and using a time corresponding to the trough point as a second time;
determining a first zero crossing point and a second zero crossing point of the pulse signal, using a time corresponding to the first zero crossing point as a third time, and using a time corresponding to the second zero crossing point as a fourth time;
determining a first threshold corresponding to the first time and the second time, and determining a second threshold corresponding to the third time and the fourth time; and
using the first threshold and the second threshold as segmentation thresholds.

In an embodiment, the determining the first threshold corresponding to the first time and the second time includes:
obtaining or determining a heart sound envelope signal corresponding to the heart sound signal;
using a heart sound peak value of the heart sound envelope signal between the first time and the second time as a first heart sound peak value; and
adjusting the first heart sound peak value based on a preset adjustment coefficient to obtain the first threshold, the first threshold is less than the first heart sound peak value.

In an embodiment, the determining the second threshold corresponding to the third time and the fourth time includes:
obtaining or determining a heart sound envelope signal corresponding to the heart sound signal;
using a heart sound peak value of the heart sound envelope signal between the third time and the fourth time as a second heart sound peak value; and
adjusting the second heart sound peak value based on a preset adjustment coefficient to obtain the second threshold, the second threshold is less than the second heart sound peak value.

In an embodiment, the segmentation threshold includes a first threshold and a second threshold, and the segmenting the heart sound signal based on the segmentation threshold includes:
obtaining or determining a heart sound envelope signal corresponding to the heart sound signal;
using a duration window in which a signal value of the heart sound envelope signal is greater than the first threshold as a first duration window;
using a duration window in which the signal value of the heart sound envelope signal is greater than the second threshold as a second duration window; and
using the heart sound signal within the first duration window as the first heart sound, and using the heart sound signal within the second duration window as the second heart sound.

In an embodiment, the determining the systolic duration and the diastolic duration of the heart sound signal according to the first heart sound and the second heart sound includes:
determining cardiac cycles included in the heart sound signal, and traversing each of the cardiac cycles sequentially;
using an end time of the first heart sound in the traversed cardiac cycle as a first end time, determining a next cardiac cycle corresponding to the traversed cardiac cycle, and using a start time of the first heart sound in the next cardiac cycle as a first start time;
using a start time of the second heart sound in the traversed cardiac cycle as a second start time, and using an end time of the second heart sound in the traversed cardiac cycle as a second end time;
using a time difference between the first end time and the second start time as the systolic duration;
using a time difference between the first start time and the second end time as the diastolic duration;
after each cardiac cycle is traversed, determining an average systolic duration of all the systolic durations and an average diastolic duration of all the diastolic durations; and
using the average systolic duration as the systolic duration of the heart sound signal, and using the average diastolic duration as the diastolic duration of the heart sound signal.

In addition, to achieve the above objectives, the present application provides a heart sound segmentation apparatus, including:
a signal acquisition module, configured to synchronously collect physiological signals, the physiological signals include heart sound signals;
a segmentation module, configured to obtain a segmentation threshold corresponding to the heart sound signal, and segment the heart sound signal based on the segmentation threshold to obtain a first heart sound and a second heart sound;
a determination module, configured to determine a systolic duration and a diastolic duration of the heart sound signal according to the first heart sound and the second heart sound.

In addition, to achieve the above objectives, the present application provides an electronic device, including:
at least one processor; and
a memory communicatively connected to the at least one processor,
the memory stores instructions that can be executed by the at least one processor, and the instructions are executed by the at least one processor to enable the at least one processor to perform the heart sound segmentation method as described above.

In addition, to achieve the above objectives, the present application provides a non-transitory computer-readable storage medium, characterized in that the readable storage medium is a computer-readable storage medium, and a program for implementing a heart sound segmentation method is stored on the computer-readable storage medium, and the program for implementing the heart sound segmentation method is executed by a processor to implement the heart sound segmentation method as described above.

In the present application, physiological signals are synchronously collected, the physiological signals include heart sound signals; a segmentation threshold corresponding to the heart sound signal is obtained, and the heart sound signal is segmented based on the segmentation threshold to obtain a first heart sound and a second heart sound; and the systolic duration and the diastolic duration of the heart sound signal are determined based on the first heart sound and the second heart sound. Thus, compared with the model-based heart sound segmentation method in the related art, the embodiment of the present application performs heart sound segmentation on the heart sound signal based on the segmentation threshold to obtain the first heart sound and the second heart sound, and calculates the diastolic duration and the systolic duration based on the first heart sound and the second heart sound, completing the complete heart sound segmentation of the heart sound signal. Only a simple value comparison operation is required to complete the heart sound segmentation, without the need for complex model operations, reducing the computational complexity, and thus reducing the resource consumption of the heart sound segmentation of the heart sound signal.

### BRIEF DESCRIPTION OF THE DRAWINGS

The accompanying drawings, which are incorporated in and constitute a part of this specification, illustrate embodiments consistent with the present application and, together with the description, serve to explain the principles of the present application.

In order to more clearly illustrate the embodiments of the present application or the technical solutions in the related art, the following briefly introduces the drawings required for use in the embodiments or the description of the related art. Obviously, for those skilled in the art, other drawings can be obtained based on these drawings without any creative work.

FIG. 1 is a flow chart of a heart sound segmentation method according to a first embodiment of the present application.

FIG. 2 is a flow chart of the heart sound segmentation method according to the first embodiment of the present application.

FIG. 3 is a schematic diagram of physiological signals of the heart sound segmentation method according to an embodiment of the present application.

FIG. 4 is a flow chart of the heart sound segmentation method according to the first embodiment of the present application.

FIG. 5 is a flow chart of the heart sound segmentation method according to a second embodiment of the present application.

FIG. 6 is a flow chart of the heart sound segmentation method according to the second embodiment of the present application.

FIG. 7 is a flow chart of the heart sound segmentation method according to the second embodiment of the present application.

FIG. 8 is a flow chart of the heart sound segmentation method according to the second embodiment of the present application.

FIG. 9 is a flow chart of the heart sound segmentation method according to a third embodiment of the present application.

FIG. 10 is a schematic diagram of an overall architecture of a wearable device according to an embodiment of the present application.

FIG. 11 is a structural schematic diagram of the wearable device according to an embodiment of the present application.

FIG. 12 is a flow chart of the heart sound segmentation method according to the third embodiment of the present application.

FIG. 13 is a flow chart of the heart sound segmentation method according to the third embodiment of the present application.

FIG. 14 is a schematic diagram of a prediction process of the heart sound segmentation method according to an embodiment of the present application.

FIG. 15 is a schematic diagram of the prediction process of the heart sound segmentation method according to another embodiment of the present application.

FIG. 16 is a schematic diagram of a heart sound segmentation apparatus according to a fourth embodiment of the present application.

FIG. 17 is a structural schematic diagram of a hardware operating environment involved in the heart sound segmentation apparatus according to a fifth embodiment of the present application.

The purpose, features and advantages of the present application will be further explained with reference to the accompanying drawings in conjunction with the embodiments.

### DETAILED DESCRIPTION OF THE EMBODIMENTS

To make the above-mentioned objects, features, and advantages of the present application more clearly understood, the technical solutions in the embodiments of the present application will be clearly and completely described below in conjunction with the accompanying drawings of the embodiments of the present application. Obviously, the described embodiments are only some of the embodiments of the present application, not all of them. Based on the embodiments of the present application, all other embodiments obtained by those skilled in the art without making any creative efforts shall fall within the scope of protection of the present application.

### First Embodiment

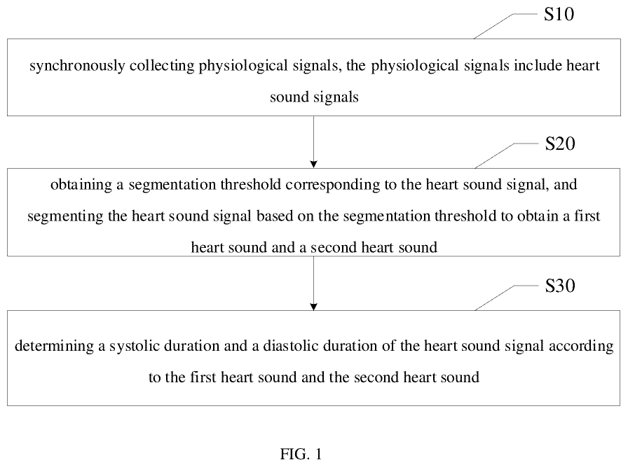

A heart sound segmentation method according to a first embodiment. As shown in FIG. 1, the heart sound segmentation method includes:

Step S10, synchronously collecting physiological signals, the physiological signals include heart sound signals.

The physiological signals include, but are not limited to, heart sound signals. For example, they may also include electrocardiogram (ECG) signals and pulse signals. The ECG and pulse signals are used to assist in the segmentation of heart sound signals, thereby improving segmentation accuracy. The physiological signals may be collected by relevant devices. For example, if a user uses a wearable device, the user's physiological signals may be collected based on the wearable device. In an embodiment, the wearable device may be a smartwatch, smart bracelet, or the like.

Furthermore, the system simultaneously starts collecting physiological signals and determines the relationship between the ECG signal, pulse signal, and heart sound signal and time to ensure strict synchronization of the physiological signals. Furthermore, the system can also record the moment the data collection starts using the verified real-time time.

Step S20, obtaining a segmentation threshold corresponding to the heart sound signal, and segmenting the heart sound signal based on the segmentation threshold to obtain a first heart sound and a second heart sound.

The heart sound segmentation involves distinguishing the exact locations of the first heart sound (S1), systole, second heart sound (S2), and diastole of each cardiac cycle. The first heart sound (S1) is caused by the closure of the mitral and tricuspid valves, the opening of the aortic and pulmonary valves, and the vibrations generated during ventricular contraction. The second heart sound (S2) is primarily caused by the closure of the aortic and pulmonary valves, coupled with vibrations caused by the reduction of blood flow in the aorta and pulmonary arteries. Systole is the interval between S1 and S2, while diastole is from S2 to the start of the next cardiac cycle S1. The accuracy of heart sound signal segmentation directly impacts the subsequent analysis of heart sound signals. The systolic duration is the duration between S1 and S2, and the diastolic duration is the duration from S2 to the start of the next cardiac cycle, S1.

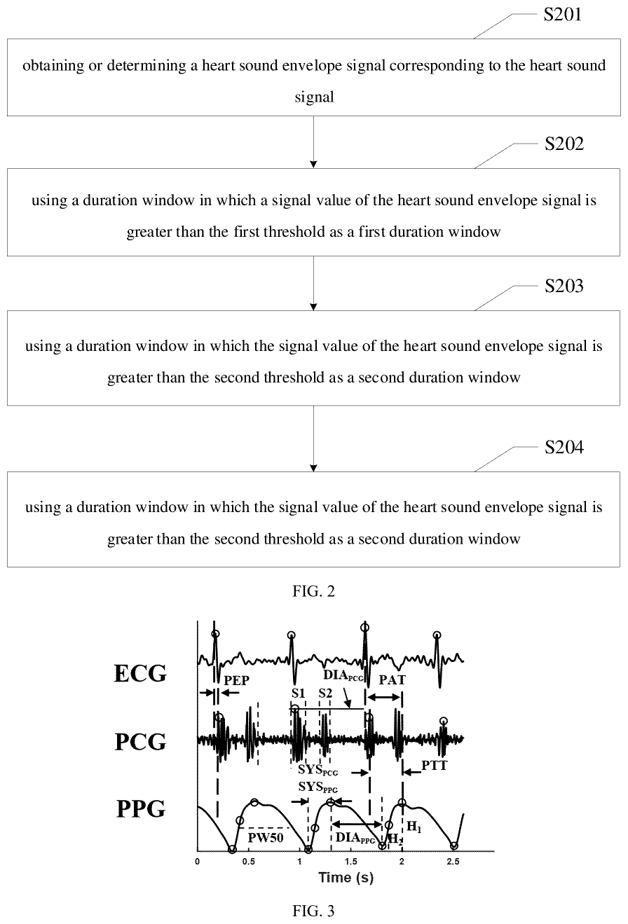

As shown in FIG. 2, in an embodiment, the segmentation threshold includes a first threshold and a second threshold, and the step of segmenting the heart sound signal based on the segmentation threshold includes:

Step S201, obtaining or determining a heart sound envelope signal corresponding to the heart sound signal.

In an embodiment, the upper envelope of the heart sound signal may be determined, and the upper envelope may be filtered to obtain a heart sound envelope signal corresponding to the heart sound signal.

Step S202, using a duration window in which a signal value of the heart sound envelope signal is greater than the first threshold as a first duration window;

Step S203, using a duration window in which the signal value of the heart sound envelope signal is greater than the second threshold as a second duration window; and

Step S204, using the heart sound signal within the first duration window as the first heart sound, and using the heart sound signal within the second duration window as the second heart sound.

It should be noted that the filtering process for the heart sound signal may be baseline removal and sliding average filtering process to reduce interference signals.

It can be understood that for each cardiac cycle, there is a corresponding first duration window and a second duration window. The first heart sound within the first duration window is denoted as S1(t), where t is the t-th cardiac cycle, and the second heart sound within the second duration window is denoted as S2(t). As shown in FIG. 3, physiological signals include electrocardiogram (ECG) signals, phonocardiogram (PCG) signals, and pulse signals. In an embodiment, the heart sound signal is segmented to obtain the first heart sound (S1) and the second heart sound (S2) within each cardiac cycle.

Step S30, determining a systolic duration and a diastolic duration of the heart sound signal according to the first heart sound and the second heart sound.

The first heart sound is S1, the second heart sound is S2, systole is the interval between S1 and S2, and diastole is the interval between S2 and the start of the next cardiac cycle, S1. In other words, the systolic duration is the interval between S1 and S2, and the diastolic duration is the interval between S2 and the start of the next cardiac cycle, S1.

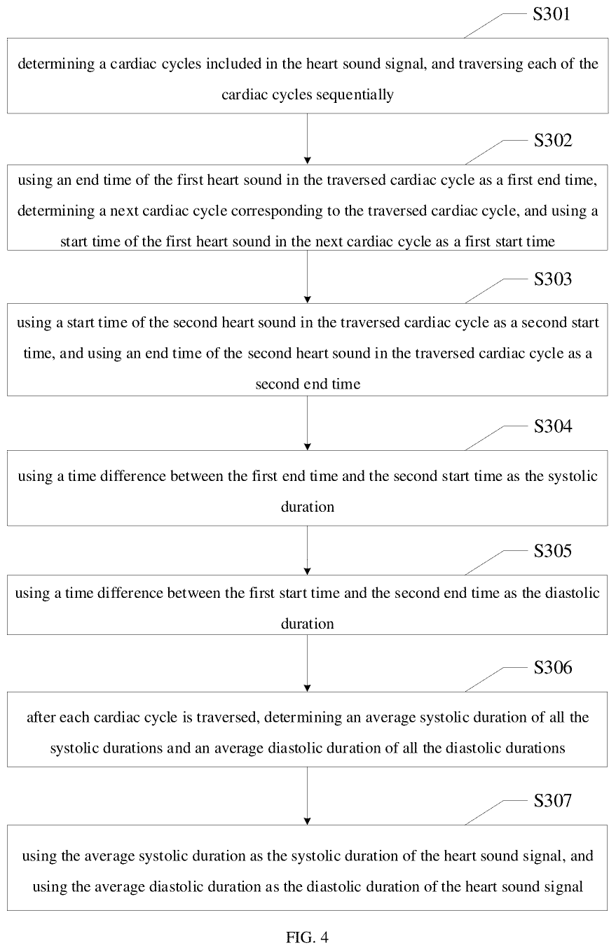

As shown in FIG. 4, in an embodiment, the step of determining the systolic duration and the diastolic duration of the heart sound signal according to the first heart sound and the second heart sound includes:

Step S301, determining a cardiac cycles included in the heart sound signal, and traversing each of the cardiac cycles sequentially;

Step S302, using an end time of the first heart sound in the traversed cardiac cycle as a first end time, determining a next cardiac cycle corresponding to the traversed cardiac cycle, and using a start time of the first heart sound in the next cardiac cycle as a first start time;

Step S303, using a start time of the second heart sound in the traversed cardiac cycle as a second start time, and using an end time of the second heart sound in the traversed cardiac cycle as a second end time;

Step S304, using a time difference between the first end time and the second start time as the systolic duration;

Step S305, using a time difference between the first start time and the second end time as the diastolic duration;

Step S306, after each cardiac cycle is traversed, determining an average systolic duration of all the systolic durations and an average diastolic duration of all the diastolic durations; and

Step S307, using the average systolic duration as the systolic duration of the heart sound signal, and using the average diastolic duration as the diastolic duration of the heart sound signal.

The first heart sound of the t-th cardiac cycle is recorded as S1(t), the second heart sound of the t-th cardiac cycle is recorded as S2(t). The starting time of S1(t) is recorded as S1ₛₜₐᵣₜ₍ₜ₎, the end time of S1(t) is recorded as S1_{end(t)}. The starting time of S2(t) is recorded as S2ₛₜₐᵣₜ₍ₜ₎, and the end time of S2(t) is recorded as S2_{end(t)}. Then the systolic duration corresponding to the t-th cardiac cycle is the time difference between S1_{end(1)} and S2ₛₜₐᵣₜ₍ₜ₎, and the diastolic duration is the time difference between S1ₛₜₐᵣₜ₍ₜ₊₁₎ and S2_{end(t)}. Based on this, if the heart sound signal includes the heart sound signals of multiple cardiac cycles, then for each cardiac cycle corresponding to a systolic duration and a diastolic duration, the average duration of the systolic duration corresponding to all cardiac cycles can be used as the final systolic duration, and similarly, the average duration of the diastolic duration corresponding to all cardiac cycles can be used as the final diastolic duration.

### Second Embodiment

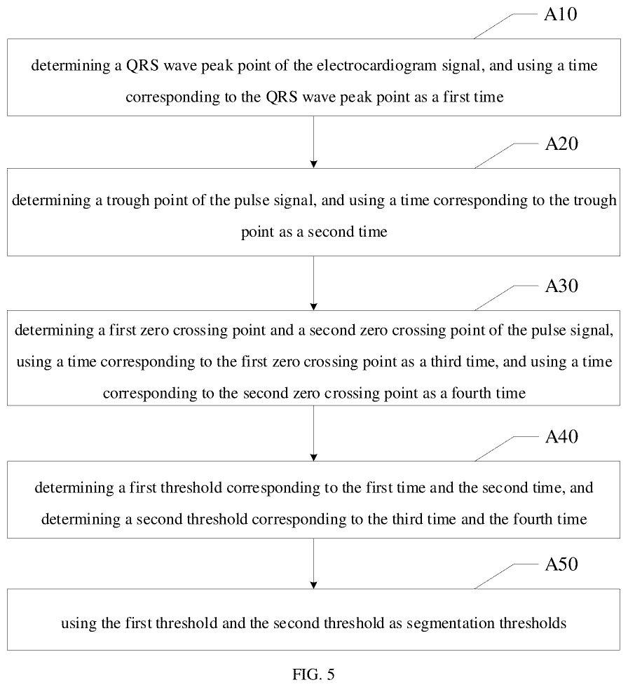

Based on the first embodiment of the present application, in an embodiment of the present application, the same or similar contents as those in the above-mentioned first embodiment can be referred to the above introduction and will not be repeated hereafter. On this basis, in an embodiment, the physiological signal also includes an electrocardiogram signal and a pulse signal, and before the step of obtaining the segmentation threshold corresponding to the heart sound signal, as shown in FIG. 5 the method further includes:
Step A10, determining a QRS wave peak point of the electrocardiogram signal, and using a time corresponding to the QRS wave peak point as a first time;
Step A20, determining a trough point of the pulse signal, and using a time corresponding to the trough point as a second time;
Step A30, determining a first zero crossing point and a second zero crossing point of the pulse signal, using a time corresponding to the first zero crossing point as a third time, and using a time corresponding to the second zero crossing point as a fourth time;
Step A40, determining a first threshold corresponding to the first time and the second time, and determining a second threshold corresponding to the third time and the fourth time; and
Step A50, using the first threshold and the second threshold as segmentation thresholds.

It can be understood that, for the electrocardiogram signal and pulse signal of each cardiac cycle, the first time corresponding to the QRS wave peak point of the electrocardiogram signal in the cardiac cycle, the second time corresponding to the trough point of the pulse signal, and the third time corresponding to the first zero crossing point and the fourth time corresponding to the second zero crossing point of the pulse signal are determined, and then the first threshold and the second threshold of the cardiac cycle are determined, and the heart sound signal of the cardiac cycle is segmented using the first threshold and the second threshold of the cardiac cycle to obtain the second systolic duration and the second diastolic duration of the heart sound signal of the cardiac cycle, thereby performing heart sound segmentation on the heart sound signal based on each cardiac cycle, thereby improving the accuracy of heart sound segmentation of the heart sound signal.

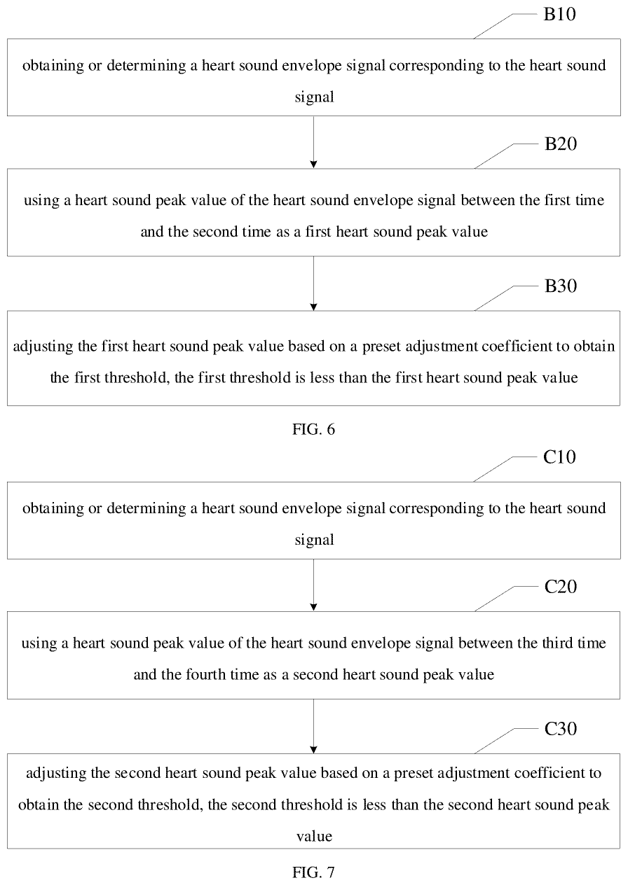

In an embodiment, as shown in FIG. 6, the step of determining the first threshold corresponding to the first time and the second time includes:
Step B10, obtaining or determining a heart sound envelope signal corresponding to the heart sound signal;
Step B20, using a heart sound peak value of the heart sound envelope signal between the first time and the second time as a first heart sound peak value; and
Step B30, adjusting the first heart sound peak value based on a preset adjustment coefficient to obtain the first threshold, the first threshold is less than the first heart sound peak value.

The preset adjustment coefficient can be any coefficient set in advance, such as 0.1, 0.15, 0.2, etc. The first heart sound peak value is adjusted based on the preset adjustment coefficient. In an embodiment, the first threshold can be obtained by multiplying the preset adjustment coefficient by the first heart sound peak value.

In the embodiment, the first threshold is determined based on the first heart sound peak value between the first time and the second time. The first threshold is correlated with the first heart sound peak value. Different peak values correspond to different threshold values, thereby realizing intelligent adjustment of the threshold value instead of using a fixed threshold value for heart sound segmentation, thereby improving the segmentation accuracy of heart sound segmentation.

As shown in FIG. 7, in an embodiment, the step of determining the second threshold corresponding to the third time and the fourth time includes:
Step C10, obtaining or determining a heart sound envelope signal corresponding to the heart sound signal;
Step C20, using a heart sound peak value of the heart sound envelope signal between the third time and the fourth time as a second heart sound peak value; and
Step C30, adjusting the second heart sound peak value based on a preset adjustment coefficient to obtain the second threshold, the second threshold is less than the second heart sound peak value.

The preset adjustment coefficient can be the same as or different from the aforementioned adjustment coefficient, and the second heart sound peak value is adjusted based on the preset adjustment coefficient. In an embodiment, the second threshold is obtained by multiplying the preset adjustment coefficient by the second heart sound peak value. The second threshold is correlated with the second peak value. Different peak values corresponding to different threshold values, this allows for intelligent adjustment of the threshold value, rather than using a fixed threshold for heart sound segmentation, thereby improving the accuracy of heart sound segmentation.

In order to help understand the technical concept or technical principle of the present application, an embodiment is listed below.

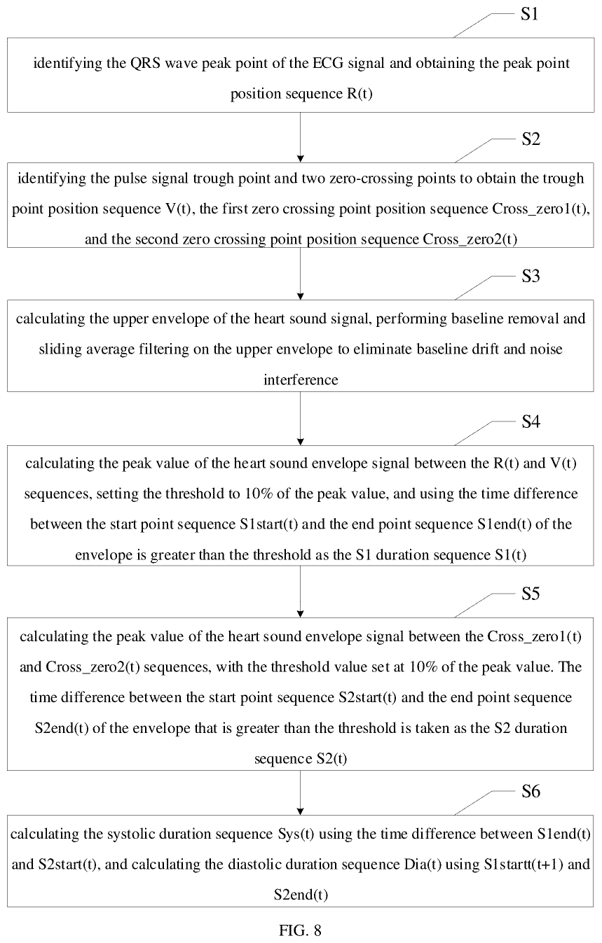

As shown in FIG. 8, in the embodiment, the heart sound segmentation process is as follows:
S1, identifying the QRS wave peak point of the ECG signal and obtaining the peak point position sequence R(t);
S2, identifying the pulse signal trough point and two zero-crossing points to obtain the trough point position sequence V(t), the first zero crossing point position sequence Cross_zero1(t), and the second zero crossing point position sequence Cross_zero2(t);
S3, calculating the upper envelope of the heart sound signal, performing baseline removal and sliding average filtering on the upper envelope to eliminate baseline drift and noise interference;
S4, calculating the peak value of the heart sound envelope signal between the R(t) and V(t) sequences, setting the threshold to 10% of the peak value, and using the time difference between the start point sequence S1ₛₜₐᵣₜ₍ₜ₎ and the end point sequence S1_{end(t)} of the envelope is greater than the threshold as the S1 duration sequence S1(t);
S5, calculating the peak value of the heart sound envelope signal between the Cross_zero1(t) and Cross_zero2(t) sequences, with the threshold value set at 10% of the peak value. The time difference between the start point sequence S2ₛₜₐᵣₜ₍ₜ₎ and the end point sequence S2_{end(t)} of the envelope that is greater than the threshold is taken as the S2 duration sequence S2(t); and
S6, calculating the systolic duration sequence Sys(t) using the time difference between S1_{end(t)} and S2ₛₜₐᵣₜ₍ₜ₎, and calculating the diastolic duration sequence Dia(t) using S1ₛₜₐᵣₜₜ₍ₜ₊₁₎ and S2_{end(t)}.

It should be noted that the above specific embodiments are only used to understand the present application and do not constitute a limitation on the heart sound segmentation process of the present application. More simple transformations based on this technical concept are all within the scope of protection of the present application.

### Third Embodiment

Blood pressure is a key physiological parameter reflecting human health. With the accelerated aging of the population and changes in modern lifestyles, the incidence of hypertension is also increasing. As of 2022, the number of people with hypertension in China reached 245 million, while the control rate and awareness of hypertension are very low, at only 56.6% and 16.8% respectively. Hypertension is a major risk factor for cardiovascular disease, the leading cause of death in the human body. Therefore, accurate and convenient blood pressure monitoring can greatly help prevent hypertension.

Traditional blood pressure measurement methods mostly rely on cuff-type electronic blood pressure monitors based on the oscillometric method. However, this method causes significant discomfort to the user due to the inflation and deflation of the cuff during measurement, and the measurement equipment is bulky and not portable. The emergence of cuffless blood pressure measurement devices in recent years has solved the problem of cuff discomfort and significantly reduced their size. With the increasing use of wearable devices in recent years, the demand for wearable blood pressure measurement devices has also increased. In particular, some cuffless measurement devices have been integrated into smartwatches, which has increased their popularity among users and the real-time measurement performance. However, the accuracy of cuffless blood pressure measurement devices currently needs to be improved.

Cuffless blood pressure measurement devices primarily estimate blood pressure through pulse wave transit time (PWTT). PWTT is defined as the time required for blood to travel from the proximal end point to the distal end point at the same instant. PWTT is typically obtained by synchronously obtaining the ECG signal and the pulse wave signal. The time difference between the R wave peak of the ECG signal and the characteristic point of the pulse wave is the PWTT.

However, the R wave peak is not actually the onset of cardiac contraction. There is a period of preparation before contraction called the pre-ejection period (PEP). This period makes blood pressure estimates based on pulse wave transit time unreliable.

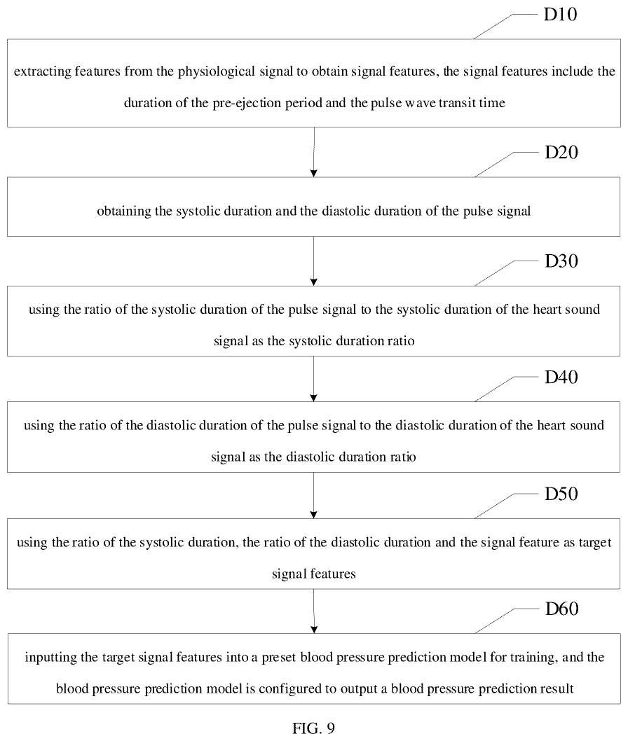

Based on the above problems, as well as the first and second embodiments of the present application, in an embodiment of the present application, the same or similar contents as those in the first, second, or third embodiments can be referred to above and will not be described in detail. On this basis, in an embodiment, after the step of determining the systolic duration and the diastolic duration of the heart sound signal according to the first heart sound and second heart sound, as shown in FIG. 9, the method further includes:
Step D10, extracting features from the physiological signal to obtain signal features, the signal features include the duration of the pre-ejection period and the pulse wave transit time.

In the embodiment, the heart sound segmentation method is applied to a wearable device, which may specifically be a smart watch, a smart bracelet, and the like.

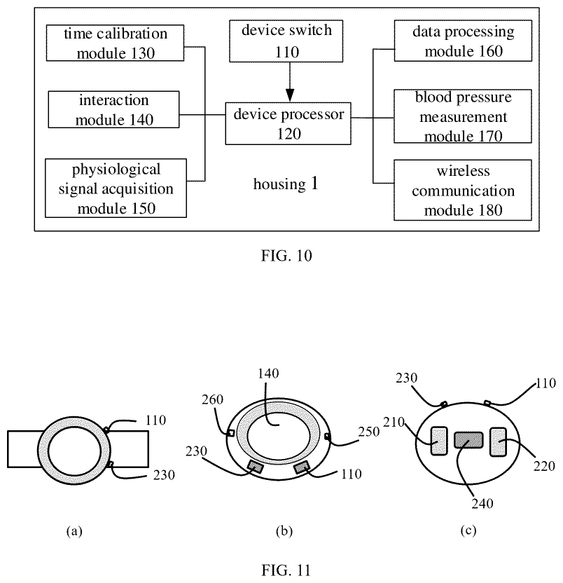

For example, as shown in FIG. 10 and FIG. 11, the wearable device includes a housing 1 and a sensor provided at the housing, and the sensor is used to synchronously collect physiological signals. The sensor includes a first sensor for collecting heart sound signals, a second sensor for collecting pulse signals, and a third sensor for collecting electrocardiogram signals. The first sensor can be a Voice Pick Up (VPU, or bone conduction) sensor 250, the second sensor can be a photoelectric pulse sensor 240, and the third sensor can be an electrode sensor. In an embodiment, the electrode sensor includes three electrodes. The first electrode 210 and the third electrode 230 are used to form a circuit for collecting electrocardiogram signals. The second electrode 220 provides a reference point to eliminate the potential difference between the body and the wearable device, thereby improving the signal-to-noise ratio of the electrocardiogram signal collection.

Furthermore, as shown in FIG. 10 and FIG. 11, the wearable device may also include: a device switch 110, a device processor 120, a time calibration module 130, an interaction module 140, a physiological signal acquisition module 150, a data processing module 160, a blood pressure measurement module 170, and a wireless communication module 180.

The device switch 110 is configured to turn the wearable device on and off.

The device processor 120 is configured to execute program code in memory to perform various functions of the wearable device.

The time calibration module 130 is configured to display real-time time and calibrate synchronously collected data.

The interaction module 140 is configured to collect personalized user information and respond to signals generated by the user performing blood pressure measurement operations, as well as simple usage instructions of blood pressure measurement. The blood pressure measurement operation signal includes a blood pressure measurement start signal, and the usage instructions include the collection location of the blood pressure measurement signal and the user's collection posture.

The physiological signal acquisition module 150 is configured to collect the user's physiological signals related to blood pressure measurement, as well as acceleration and gyroscope signals. Physiological signals related to blood pressure measurement include electrocardiogram signals, pulse signals, and heart sound signals.

The data processing module 160 is configured to process the physiological signal segments collected by the physiological signal measurement module in real time. The data processing steps mainly include signal noise reduction, signal quality assessment, and feature extraction.

The blood pressure measurement module 170 is configured to analyze the user's physiological signal data and/or personalized information, ultimately predict blood pressure, return the predicted blood pressure result, and determine the current blood pressure level.

The wireless communication module 180 is configured to transmit collected physiological signal data, the user's actual blood pressure value, and personalized user information to a server or terminal through a wireless transmission module. These data is configured to build a blood pressure measurement database.

In addition, as shown in FIG. 11, the physiological signal acquisition module also includes a 6-axis signal acquisition module. Acceleration and gyroscope signals are primarily acquired through the 6-axis signal acquisition module, and the 6-axis signal acquisition module is primarily composed of a 6-axis sensor 260 integrated within the device. Before a blood pressure measurement is performed, the 6-axis signal acquisition module can calculate the current Euler angle to assist the user in locating the acquisition position and posture. During the blood pressure measurement process, the 6-axis signal acquisition module can monitor the movement of the user's arm and combine it with the electrocardiogram (ECG) signal, heart sound signal, and pulse signal to perform physiological signal denoising, thereby improving the signal-to-noise ratio of these signals during the acquisition process.

It is understood that the structures illustrated in the embodiments of the present application do not constitute a specific limitation on the wearable device. It may have more or fewer of the above components, or combine or separate some components, or have different component arrangements. The various components described above may be implemented in hardware, software, or a combination of hardware and software, including one or more signal processing or application-specific integrated circuits.

The test subject can be a user using the wearable device. Furthermore, simultaneous physiological signal acquisition begins at the same instant, the relationship between the ECG signal, pulse signal, and heart sound signal and time is determined to ensure strict synchronization of the physiological signals. Furthermore, the data can be recorded from the moment acquisition begins, using the calibrated real-time time.

Furthermore, before the extracting features from the physiological signal, to improve the accuracy of the extracted signal features, the physiological signal may be processed, such as filtering, noise reduction, segmentation, etc. Feature extraction is performed based on the processed physiological signal to obtain the signal features.

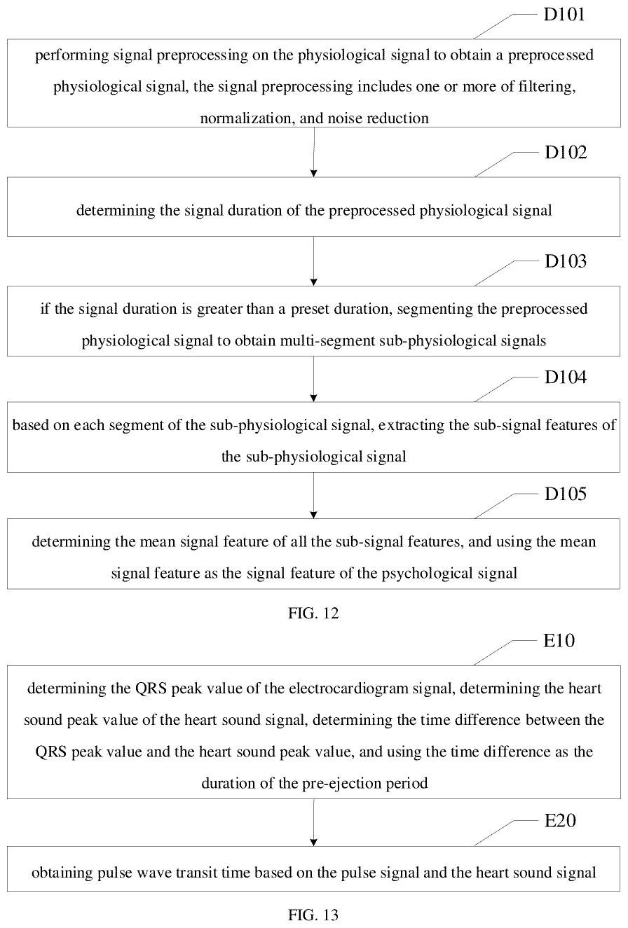

In an embodiment, as shown in FIG. 12, the step of extracting features from the physiological signal includes:
Step D101, performing signal preprocessing on the physiological signal to obtain a preprocessed physiological signal, the signal preprocessing includes one or more of filtering, normalization, and noise reduction;

In the embodiment, signal preprocessing includes normalization, filtering, and noise reduction. In an embodiment, the collected physiological signals are normalized and then bandpass filtered using a pre-designed finite impulse response (FIR) bandpass filter on the ECG, pulse, and heart sound signals. The filtered data and acceleration signals are then subjected to a secondary adaptive filter to remove noise caused by motion artifacts, thereby obtaining clean and high-quality physiological signals.

Step D102, determining the signal duration of the preprocessed physiological signal.

Step D103, if the signal duration is greater than a preset duration, segmenting the preprocessed physiological signal to obtain multi-segment sub-physiological signals.

It should be noted that the preprocessed physiological signal is segmented to obtain multi-segment sub-physiological signals. The physiological signal can be segmented without temporal overlap. For example, if the signal duration is 12 seconds and the signal is segmented with a duration of 3 seconds, it can be divided into four sub-physiological signals: 0-3 seconds, 3-6 seconds, 6-9 seconds, and 9-12 seconds. In an embodiment, the physiological signal can be segmented with temporal overlap, where each signal is divided into signal segments of a fixed duration (Sig_t), with the overlap length being any value in the range (0, Sig_t−1). For example, if the signal duration is 9 seconds and the signal is segmented with a fixed duration of 3 seconds and an overlap length of 1 second, it can be divided into four sub-physiological signals: 0-3 seconds, 2-5 seconds, 4-7 seconds, and 6-9 seconds.

Furthermore, after the preprocessed physiological signal is segmented to obtain multi-segment sub-physiological signals, the signal quality of each sub-physiological signal can be evaluated, the sub-physiological signals with poor signal quality can be removed, and the signal features can be extracted based on the sub-physiological signals with good signal quality to ensure the validity of the extracted signal features.

In an embodiment, the signal quality of each sub-physiological signal segment can be assessed by calculating features such as the RR interval and KSQI index of the ECG signal, the peak interval and number of zero crossings of the pulse signal, and the SSQI coefficient and signal mean of the heart sound signal. These features are then combined with the time variation of the acceleration signal amplitude (e.g., excluding sub-physiological signals with temporal overlap with the acceleration signal amplitude) to eliminate signal segments affected by noise. These features are then fed into a classification model, such as a Support Vector Machine (SVM), to perform a rough classification of each signal segment, such as into usable and unusable sub-physiological signals, and extract signal features based on the usable sub-physiological signals.

Step D104, based on each segment of the sub-physiological signal, extracting the sub-signal features of the sub-physiological signal.

Step D105, determining the mean signal feature of all the sub-signal features, and using the mean signal feature as the signal feature of the psychological signal.

If the duration of the preprocessed physiological signal is longer than a preset duration, such as 5 seconds, 6 seconds, 7 seconds, etc., the preprocessed physiological signal may be segmented to extract sub-signal features of each sub-physiological signal.

It can be understood that the preprocessed physiological signals are segmented, that is, the preprocessed ECG signal, the preprocessed pulse signal, and the preprocessed heart sound signal are segmented. The sub-signal features of each sub-physiological signal are extracted, and each sub-signal feature constitutes an n-dimensional feature sequence X(t), where n is the number of features. The mean of each dimensional feature of all X(t) is calculated to obtain the mean signal feature, which is the final signal feature. For example, assuming that the psychological signal physiological signal includes an ECG signal, a pulse signal, and a heart sound signal, after segmentation processing, three sub-physiological signals are obtained, namely sub-physiological signal 1, sub-physiological signal 2, and sub-physiological signal 3. Sub-physiological signal 1 includes sub-ECG signal 1, sub-heart sound signal 1, and sub-pulse signal 1. Sub-physiological signal 2 includes sub-ECG signal 2, sub-heart sound signal 2, and sub-pulse signal 2. Sub-physiological signal 3 includes sub-ECG signal 3, sub-heart sound signal 3, and sub-pulse signal 3. The signal features of sub-physiological signal 1 are extracted and recorded as sub-signal feature 1 [1_1, 1_2, 1_3], the signal features of sub-physiological signal 1 are extracted and recorded as sub-signal feature 2 [2_1, 2_2, 2_3], and the signal features of sub-physiological signal 3 are extracted and recorded as sub-signal feature 3 [3_1, 3_2, 3_3]. The final signal features are [(1_1+2_1+3_1)/3, (1_2+2_2+3_2)/3, (1_3+2_3+3_3)/3].

Furthermore, after extracting the sub-signal features of each sub-physiological signal, outlier processing can be performed on all sub-signal features to remove abnormal features. In an embodiment, for the signal features of each sub-physiological signal segment, outlier feature sequence processing can be performed according to the boxplot principle. In an embodiment, a processing flow can be as follows: (a), based on the feature values of each sub-physiological signal segment, forming an n-dimensional feature sequence X(t), where n is the number of features; (b), setting the upper and lower edges of each feature dimension, such as calculating the upper quartile Q1i, lower quartile Q3i, and interquartile range IQRi of each feature dimension, using the upper edge Q1ᵢ−1.5IQRᵢ and the lower edge Q3ᵢ+1.5IQRᵢ, where i=1:n; (c), filtering out abnormal data outside the upper and lower edges of each feature dimension; (d), obtaining the feature sequence S(t) after removing outliers; and (e), calculating the mean of the filtered feature sequence to obtain the mean signal feature S-.

The signal characteristics include but are not limited to the duration of the pre-ejection period and pulse wave transit time, such as pulse arrival time (PAT), the systolic duration ratio of the pulse signal and the heart sound signal, the diastolic duration ratio of the pulse signal and the heart sound signal, time domain features, frequency domain features, time-frequency features, statistical features, etc.

In an embodiment, time domain features include but are not limited to the following features: R-R interval (RR), R-R standard deviation (SDNN), root mean square deviation (RMSD) of ECG signals; PP interval (PP), half pulse width (PW50), systolic duration, diastolic duration, rise duration, fastest rise area, peak height, rise slope, etc. of pulse signals; first zero crossing time, last inflection point time, peak and first zero crossing slope, peak slope, peak area, etc. of Velocity Plethysmogram (VPG) signals (obtained by first-order differentiation of pulse signals); lowest point time, peak and lowest point slope, first zero crossing time and lowest point slope of Acceleration Plethysmogram (APG) signals (obtained by second-order differentiation of pulse signals); first heart sound duration, second heart sound duration, systolic duration, diastolic duration, etc. of heart sound signals.

Frequency domain features include but are not limited to the following features: power spectrum density of ECG signal; first component frequency and amplitude of pulse signal, second component frequency and amplitude, third component frequency and amplitude; main component frequency of heart sound signal S1, main component frequency of S2, etc.

Time-frequency features mainly include but are not limited to the following features: wavelet coefficients, Hilbert-Huang transform coefficients, Mel-frequency cepstral coefficients, linear prediction coefficient features, etc.

Statistical features include but are not limited to the following features: kurtosis factor, skewness factor, standard deviation of characteristic sequence, etc.

Step D20, obtaining the systolic duration and the diastolic duration of the pulse signal.

The signal characteristics of the pulse signal are different in the systolic period and the diastolic period. Based on this, the systolic duration and the diastolic duration are extracted from the pulse signal. The specific extraction method can adopt the related art and will not be repeated in the embodiment.

Step D30, using the ratio of the systolic duration of the pulse signal to the systolic duration of the heart sound signal as the systolic duration ratio.

Step D40, using the ratio of the diastolic duration of the pulse signal to the diastolic duration of the heart sound signal as the diastolic duration ratio.

Step D50, using the ratio of the systolic duration, the ratio of the diastolic duration and the signal feature as target signal features.

Step D60, inputting the target signal features into a preset blood pressure prediction model for training, and the blood pressure prediction model is configured to output a blood pressure prediction result.

The trained blood pressure prediction model may be a blood pressure prediction model trained based on a database, the database includes at least real blood pressure data and signal feature data corresponding to each real blood pressure data. The database may be obtained by pre-collecting the real blood pressure data and signal feature data.

Furthermore, in order to improve the prediction accuracy of the blood pressure prediction model and realize personalized prediction of blood pressure, the real blood pressure values and physiological signals of the test subjects can be collected multiple times in advance to establish an individual data set, and the individual data set can be used as the database for training the prediction model to complete the training of the blood pressure prediction model.

In an embodiment, the pre-training process for a blood pressure prediction model can be as follows: S1, selecting features from a multi-category feature database using an existing database; S2, calculating the mutual information between each feature, where p(x) is the probability of x occurring, p(y) is the probability of y occurring, and p(x, y) is the probability of both x and y occurring simultaneously, i.e., the joint probability. Higher mutual information indicates a greater degree of dependency between the two features. The features below the mutual information threshold is removed to obtain a new feature subset; S3, calculating the correlation coefficient between the new feature subset and blood pressure. The higher the correlation coefficient, the higher the linear correlation between the feature and blood pressure; S4, sorting the feature subset from high to low according to the correlation coefficient to obtain the sorted feature subset; S5, dividing the sorted feature subset into training set and test set, with the ratio of training set to test set being 8:2; S6, for the training set, using ten-fold cross-validation and backward feature selection to select the number of features to obtain the feature subset S with the lowest RMSE (Root Mean Squared Error); S7, using the final feature subset to train the multivariate linear regression model

$$BP = {\sum }_{i = 1}^{n} {K}_{i} * {S}_{i} ,$$

where BP is the specific blood pressure value, S is the optimal feature subset, Kᵢ is the fitting coefficient of the multivariate linear regression model, and n is the dimension of the optimal feature subset.

In addition, personalized features such as the tester's age, gender, height, weight, body mass index (BMI), etc. can be collected and used for model training with signal feature data to obtain a pre-trained personalized blood pressure prediction model. After extracting signal features based on physiological signals, personalized features such as the tester's age, gender, height, weight, BMI, etc. are further tested. The signal features and personalized features are input into the pre-trained personalized blood pressure prediction model to output a blood pressure prediction result. using into account the impact of factors such as age, gender, height, weight, BMI on blood pressure, the accuracy of blood pressure prediction can be further improved.

Furthermore, after obtaining a blood pressure prediction result, it can be output on the wearable device and uploaded to a terminal or server connected to the wearable device for the user to view. The terminal and server can also view the user's historical blood pressure data, which helps doctors make diagnoses. Customizing a personalized blood pressure measurement plan based on historical data helps users more comprehensively understand their health status and provide more scientific medical advice.

In the embodiment, physiological signals of the test subject are collected, the physiological signals include electrocardiogram signals, pulse signals, and heart sound signals. Feature extraction is performed on the physiological signals to obtain signal features, the signal features include duration of the pre-ejection period and pulse wave transit time; and the signal features are input into a pre-trained blood pressure prediction model, so that the blood pressure prediction model outputs a blood pressure prediction result. Thus, compared with the related art method of estimating blood pressure based on pulse wave transit time, the embodiment predicts the test subject's blood pressure based on fused signal features such as duration of the pre-ejection period and pulse wave transit time, using into account the impact of the duration of the pre-ejection period on blood pressure, thereby improving the accuracy of blood pressure prediction.

In the embodiment, after performing heart sound segmentation on the heart sound signal, the second systolic duration and the second diastolic duration based on the heart sound signal are obtained, and the first systolic duration and the first diastolic duration of the pulse signal are obtained. The ratio between the two is also used as a signal feature, thereby comprehensively measuring the diastolic duration and systolic duration of the heart sound signal and the pulse signal, which can further improve the accuracy of blood pressure prediction.

In an embodiment, as shown in FIG. 13, the step of extracting features from the physiological signal includes:
Step E10, determining the QRS peak value of the electrocardiogram signal, determining the heart sound peak value of the heart sound signal, determining the time difference between the QRS peak value and the heart sound peak value, and using the time difference as the duration of the pre-ejection period.

It should be noted that if the physiological signal is segmented, then in the embodiment, feature extraction is performed on the physiological signal. In fact, feature extraction can be performed on each sub-physiological signal. The electrocardiogram signal, heart sound signal and pulse signal also include the electrocardiogram signal, heart sound signal and pulse signal in the sub-physiological signal.

As shown in FIG. 3, physiological signals include an ECG signal, a phonocardiogram (PCG) signal, and a pulse signal. The peak values of the ECG signal and the heart sound signal are determined. In an embodiment, the QRS peak value of the ECG signal and the heart sound peak value of the heart sound signal belonging to the same cardiac cycle are determined, and the times corresponding to these two signal peaks are obtained. The time difference between the two is used as the duration of the pre-ejection period. If the physiological signal includes multiple cardiac cycles, the duration of the pre-ejection period corresponding to each cardiac cycle can be determined, and the average of all duration of the pre-ejection periods is used as the final duration of the pre-ejection period.

Step E20, obtaining pulse wave transit time based on the pulse signal and the heart sound signal.

The S1 peak point of the heart sound signal is the starting point, and the pulse wave feature point is the end point. The time difference between the two is the pulse wave transit time. Thus, one or more pulse wave feature points can be selected to obtain the corresponding one or more pulse wave transit times. That is, the quantity of the pulse wave transit time can be one or more than one. The user can set the selection rules of the pulse wave feature points according to actual conditions. The embodiment does not impose a specific limit on the quantity of pulse wave transit time features.

Similarly, based on the synchronously collected ECG signals and pulse signals, one or more pulse arrival times (PATs) are determined with the R wave peak of the ECG signal as the starting point, and these one or more pulse arrival times can also be used as extracted signal features.

In the embodiment, fusion signal features such as the duration of the pre-ejection period and the pulse wave transit time are extracted to provide an effective data basis for predicting blood pressure values.

In order to help understand the technical concept or technical principle of the present application, an embodiment is listed below.

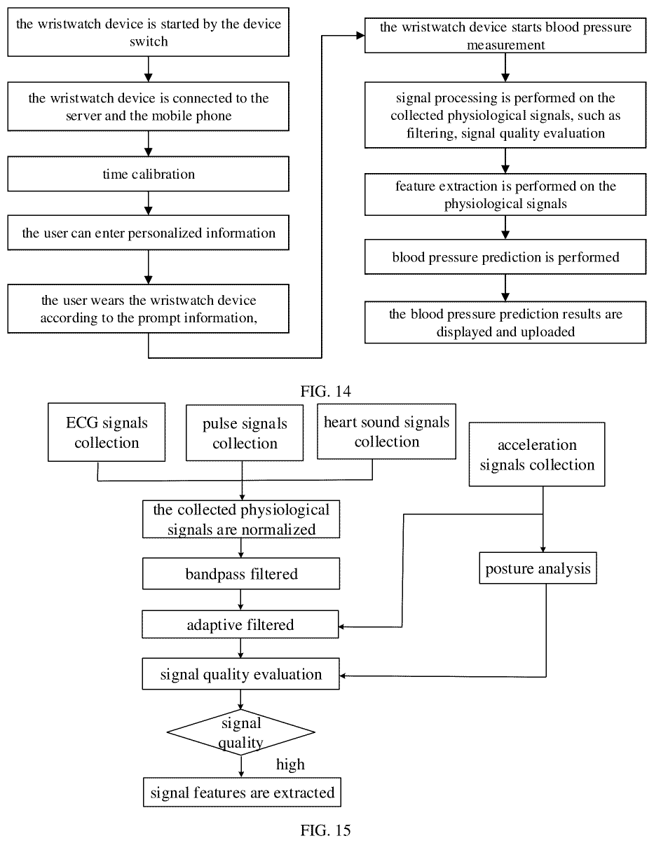

As shown in FIG. 14 and FIG. 15, the blood pressure prediction process in the embodiment is as follows.

The wristwatch device is started by the device switch. At the same time, the wristwatch device is connected to the server and the mobile phone APP. After time calibration, the user can enter personalized information, such as height, weight, etc., and the user is prompted to configure the information. After the user wears the wristwatch device according to the prompt information, the wristwatch device starts blood pressure measurement and synchronously collects physiological signals.

The physiological signals include electrocardiogram signals, heart sound signals and pulse signals. At the same time, the user's acceleration, angular velocity and other information are collected to calculate the user's current Euler angle to assist the user in locating the collection position and posture. During the blood pressure measurement process, the user's acceleration and angular velocity information is collected to monitor the movement of the user's arm, and combined with the electrocardiogram signals, heart sound signals and pulse signals to denoise the physiological signals. Signal processing is performed on the collected physiological signals, such as filtering (specifically, it may include bandpass filtering and adaptive filtering), signal quality evaluation, etc., and feature extraction is performed on the physiological signals whose signal quality evaluation results are high. The extracted signal features are input into the blood pressure prediction model to perform blood pressure prediction, and the blood pressure prediction results are displayed and uploaded at the same time.

It should be noted that the above specific embodiments are only used to understand the present application and do not constitute a limitation on the blood pressure prediction process of the present application or the application equipment. More simple transformations based on the technical concept are all within the scope of protection of the present application.

### Fourth Embodiment

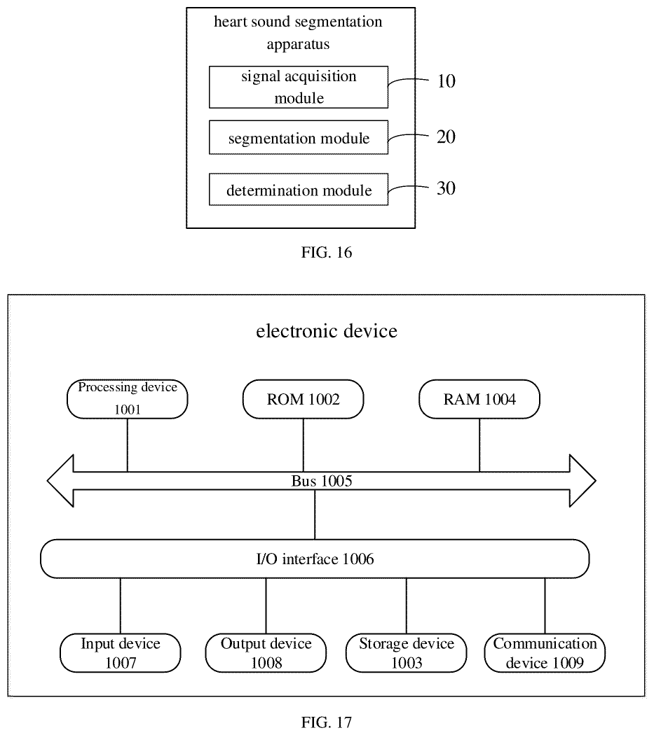

An embodiment of the present application further provides a heart sound segmentation apparatus. As shown in FIG. 16, the heart sound segmentation apparatus includes: a signal acquisition module, a segmentation module and a determination module.

The signal acquisition module is configured to synchronously collect physiological signals, the physiological signals include heart sound signals.

The segmentation module is configured to obtain a segmentation threshold corresponding to the heart sound signal, and segment the heart sound signal based on the segmentation threshold to obtain a first heart sound and a second heart sound.

The determination module is configured to determine a systolic duration and a diastolic duration of the heart sound signal according to the first heart sound and the second heart sound.

The physiological signal further includes an electrocardiogram signal and a pulse signal. The segmentation module is further configured to: determine a QRS wave peak point of the electrocardiogram signal, and use a time corresponding to the QRS wave peak point as a first time; determine a trough point of the pulse signal, and use a time corresponding to the trough point as a second time; determine a first zero crossing point and a second zero crossing point of the pulse signal, use a time corresponding to the first zero crossing point as a third time, and use a time corresponding to the second zero crossing point as a fourth time; determine a first threshold corresponding to the first time and the second time, and determine a second threshold corresponding to the third time and the fourth time; and use the first threshold and the second threshold as segmentation thresholds.

The segmentation module is further configured to: obtain or determine a heart sound envelope signal corresponding to the heart sound signal; take a heart sound peak value of the heart sound envelope signal between the first time and the second time as a first heart sound peak value; and adjust the first heart sound peak value based on a preset adjustment coefficient to obtain the first threshold, the first threshold is less than the first heart sound peak value.

The segmentation module is further configured to: obtain or determine a heart sound envelope signal corresponding to the heart sound signal;
take a heart sound peak value of the heart sound envelope signal between the third time and the fourth time as a second heart sound peak value; and
adjust the second heart sound peak value based on a preset adjustment coefficient to obtain the second threshold, the second threshold is less than the second heart sound peak value.

The segmentation threshold includes a first threshold and a second threshold, and the segmentation module is further configured to:
obtain or determine a heart sound envelope signal corresponding to the heart sound signal;
take a duration window in which a signal value of the heart sound envelope signal is greater than the first threshold as a first duration window;
take a duration window in which the signal value of the heart sound envelope signal is greater than the second threshold as a second duration window; and
use the heart sound signal within the first duration window as the first heart sound, and use the heart sound signal within the second duration window as the second heart sound.

The determination module is further configured to:
determine a cardiac cycles included in the heart sound signal, and traverse each of the cardiac cycles sequentially;
take an end time of the first heart sound in the traversed cardiac cycle as a first end time, determine a next cardiac cycle corresponding to the traversed cardiac cycle, and take a start time of the first heart sound in the next cardiac cycle as a first start time;
take a start time of the second heart sound in the traversed cardiac cycle as a second start time, and take an end time of the second heart sound in the traversed cardiac cycle as a second end time;
take a time difference between the first end time and the second start time as the systolic duration;
use a time difference between the first start time and the second end time as the diastolic duration;
after each cardiac cycle is traversed, determine an average systolic duration of all the systolic durations and an average diastolic duration of all the diastolic durations; and
use the average systolic duration as the systolic duration of the heart sound signal, and use the average diastolic duration as the diastolic duration of the heart sound signal.

The heart sound segmentation device further includes a blood pressure prediction module, the blood pressure prediction module is configured to:
extract features from the physiological signal to obtain signal features, the signal features include the duration of the pre-ejection period and the pulse wave transit time;
obtain the systolic duration and the diastolic duration of the pulse signal;
take the ratio of the systolic duration of the pulse signal to the systolic duration of the heart sound signal as the systolic duration ratio;
take the ratio of the diastolic duration of the pulse signal to the diastolic duration of the heart sound signal as the diastolic duration ratio;
take the ratio of the systolic duration, the ratio of the diastolic duration and the signal feature as target signal features; and
input the target signal features into a preset blood pressure prediction model for training, and the blood pressure prediction model is configured to output a blood pressure prediction result.

The blood pressure prediction model is also configured to:
determine the QRS peak value of the electrocardiogram signal, determine the heart sound peak value of the heart sound signal, determine the time difference between the QRS peak value and the heart sound peak value, and use the time difference as the duration of the pre-ejection period; and
obtain pulse wave transit time based on the pulse signal and the heart sound signal.

In addition, the physiological signals further include acceleration and gyroscope signals during the measurement process.

The signal acquisition module includes: an ECG measurement module, a pulse measurement module, a heart sound signal measurement module and a 6-axis signal acquisition module.

ECG signals are primarily acquired through the ECG measurement module, the ECG measurement module includes a high-impedance chip and three highly conductive dry electrodes. Pulse signals are also primarily acquired through the pulse measurement module, the pulse measurement module primarily includes a multi-wavelength light-emitting diode (LED), a photoelectric sensor, and a filter and amplifier circuit. The final pulse signal is a fusion of the multi-wavelength LEDs. To maintain the volume of the cuffless blood pressure watch, the high-impedance characteristics of the simulated ECG signal acquisition, pulse signal fusion, filter and amplifier circuitry, and synchronization of the two physiological signals are all implemented using an integrated Analog Front-End (AFE) chip.

The heart sound signal is mainly collected through the heart sound signal measurement module. The heart sound signal measurement module is mainly composed of a digital VPU sensor. The VPU sensor is attached to the inside of the watch case through a strict rigid connection method. The amplification factor of the VPU sensor is adjusted to prevent signal overflow during the test to obtain a complete heart sound signal.

Acceleration and gyroscope signals are primarily acquired through the 6-axis signal acquisition module, and the 6-axis signal acquisition module is primarily composed of a 6-axis sensor 260 integrated within the device. Before a blood pressure measurement is performed, the 6-axis signal acquisition module can calculate the current Euler angle to assist the user in locating the acquisition position and posture. During the blood pressure measurement process, the 6-axis signal acquisition module can monitor the movement of the user's arm and combine it with the ECG signal, heart sound signal, and pulse signal to perform physiological signal denoising, thereby improving the signal-to-noise ratio of these signals during the acquisition process.

In addition, the heart sound segmentation apparatus also includes: a watch housing, a time calibration module, an interaction module, a data processing module and a wireless communication module.

The watch housing is configured to house sensors for collecting physiological signals and the watch control system.

The time calibration module is configured to display real-time time and synchronize data acquisition and calibration.

The interaction module is configured to collect personalized user information, respond to signals generated by the user performing blood pressure measurement operations, and provide simple instructions for using the blood pressure watch. The signals generated by the blood pressure measurement operation include a blood pressure measurement start signal. The instructions for using the watch include the blood pressure measurement watch signal collection location and the user's collection posture.

The data processing module is configured to process the physiological signal segments collected by the physiological signal measurement module in real time. The data processing steps mainly include signal noise reduction, signal quality assessment and feature extraction.

The wireless communication module is configured to transmit the collected physiological signal data, the real user blood pressure value and the user's personalized information to the server or mobile terminal for the construction of the blood pressure measurement watch database.

The exterior of the watch housing includes the watch's switch and three dry electrodes for ECG monitoring. The watch's switch primarily controls the watch's on/off function and accesses the watch's internal menu. The first and third electrodes form a circuit for ECG signal acquisition, while the second electrode provides a reference point to eliminate the potential difference between the body and the watch, thereby improving the signal-to-noise ratio of ECG signal acquisition. The interior of the watch housing includes a rigidly connected VPU sensor and a photoelectric pulse sensor for measuring heart sound signals and photoplethysmography signals.

The time calibration module generates a real-time clock, controls the sensors to simultaneously start collecting physiological signals, and determines the relationship between the ECG signal, pulse signal, and heart sound signal and time to ensure strict synchronization of physiological signals. The calibrated real-time time is then used to record the moment of data collection after collection begins.

The interaction module includes an input module, a display module, and a prompt module, the input module, the display module, and the prompt module are implemented by the watch's internal microcontroller unit (MCU) and the watch screen. Input module 141 within the interaction module is used to input personalized information into the watch through the watch screen before using a blood pressure measurement. The information includes not only personal information such as gender, height, age, and weight, but also information such as whether or not a person is using antihypertensive medication and the name of the medication.

The display module in the interaction module is used to display how to use the watch and the measurement steps. After completing the personalized information input step, the user can click on the blood pressure measurement function on the screen to see how to wear the watch and the posture the user should maintain during the measurement. After completing the usage tutorial, the user will enter the testing phase. The watch can prompt the user to adjust the arm position through the prompt module to ensure the quality of physiological signal acquisition.

The prompt module in the interaction module includes a voice chip and a linear vibration motor, the prompt module is used to prompt the operation process, arm position and measurement posture to ensure the accuracy of blood pressure measurement.

The data processing module includes: a signal noise reduction module and a signal quality evaluation module.

The signal noise reduction module normalizes the collected physiological signals and then applies bandpass filtering to the ECG, pulse, and heart sound signals using a pre-designed FIR bandpass filter. The filtered data and acceleration signals are then processed again using adaptive filtering to remove noise caused by motion artifacts and obtain clean, high-quality physiological signals.

The signal quality evaluation module divides each signal into segments of a fixed duration (Sig_t), typically >5 seconds, with an overlap length ranging from 0 to Sig_t−1. For each segment, it calculates features such as the RR interval and KSQI index for the ECG signal; the peak interval, peak interval standard deviation, and number of zero crossings for the pulse signal; and the SSQI coefficient and signal mean for the heart sound signal. Combined with the time-varying amplitude of the acceleration signal, the SVM model is used to perform a rough classification of each segment, thereby eliminating segments affected by noise.

The wireless communication module is used to send the collected physiological signals, input personalized information and the actual measured blood pressure value to the terminal and upload them to the cloud server for data collection.

The heart sound segmentation apparatus provided by the present application, employing the heart sound segmentation method described in the first, second, or third embodiments, can address the technical problem of reducing resource consumption in heart sound segmentation. Compared to the related art, the heart sound segmentation apparatus provided by the present application achieves the same beneficial effects as the heart sound segmentation method described in the aforementioned embodiments. Other technical features of the heart sound segmentation apparatus are the same as those disclosed in the aforementioned embodiments and are not further elaborated here.

### Fifth Embodiment

An embodiment of the present application provides an electronic device, including: at least one processor; and a memory communicatively connected to the at least one processor; the memory stores instructions executable by the at least one processor, and the instructions are executed by the at least one processor so that the at least one processor can execute the heart sound segmentation method in the above-mentioned first embodiment.

As shown in FIG. 17, which illustrates a structural schematic diagram of an electronic device suitable for implementing embodiments of the present application. The electronic device in the embodiments of the present application may be a wearable device. The electronic device illustrated in FIG. 17 is merely an example and should not limit the functionality or scope of use of the embodiments of the present application.

As shown in FIG. 17, the electronic device may include a processing device 1001 (e.g., a central processing unit, a graphics processing unit, etc.), which can perform various appropriate actions and processes based on programs stored in a read-only memory (ROM 1002) or programs loaded from a storage device into a random access memory (RAM 1004). RAM 1004 also stores various programs and data required for the operation of the electronic device. Processing device 1001, ROM 1002, and RAM 1004 are interconnected via a bus 1005. An input/output (I/O) interface is also connected to bus 1005.

Typically, the following systems may be connected to I/O interface 1006: input device 1007 including, for example, a touch screen, touchpad, keyboard, mouse, image sensor, microphone, accelerometer, gyroscope, etc.; output device 1008 including, for example, a liquid crystal display (LCD), speaker, vibrator, etc.; storage device 1003 including, for example, a magnetic tape, hard disk, etc.; and communication device 1009. Communication device 1009 may allow the electronic device to communicate with other devices wirelessly or by wire to exchange data. Although the figure shows an electronic device with various systems, it should be understood that not all of the illustrated systems are required to be implemented or present. More or fewer systems may alternatively be implemented or present.

In particular, according to an embodiment of the present application, the process described above with reference to the flowchart can be implemented as a computer software program. For example, an embodiment of the present application includes a computer program product, which includes a computer program carried on a computer-readable medium, and the computer program includes program code for executing the method shown in the flowchart. In such an embodiment, the computer program can be downloaded and installed from a network via a communication device, or installed from a storage device 1003, or installed from a ROM 1002. When the computer program is executed by the processing device 1001, the above-mentioned functions defined in the method of the embodiment of the present application are performed.

The electronic device provided by the present application adopts the heart sound segmentation method described in the above-mentioned embodiment, which can solve the technical problem of reducing resource consumption in heart sound segmentation. Compared to the related art, the electronic device provided by the present application achieves the same beneficial effects as the heart sound segmentation method described in the above-mentioned embodiment. Other technical features of the electronic device are the same as those disclosed in the above-mentioned embodiment and are not further elaborated here.

It should be understood that various parts of the present application can be implemented with hardware, software, firmware or a combination thereof. In the description of the above embodiments, specific features, structures, materials or characteristics can be combined in any one or more embodiments or examples in an appropriate manner.

The above description is merely a specific embodiment of the present application, but the scope of protection of the present application is not limited thereto. Any modifications or substitutions that can be easily conceived by a person skilled in the art within the technical scope disclosed in the present application should be included in the scope of protection of the present application. Therefore, the scope of protection of the present application should be based on the scope of protection of the claims.

### Sixth Embodiment

An embodiment of the present application provides a computer-readable storage medium having computer-readable program instructions stored thereon, and the computer-readable program instructions are used to execute the heart sound segmentation method in the above-mentioned first embodiment.

The computer-readable storage medium provided in the embodiments of the present application may be, for example, a USB flash drive, but is not limited to electrical, magnetic, optical, electromagnetic, infrared, or semiconductor systems, systems, or devices, or any combination thereof. More specific examples of computer-readable storage media may include, but are not limited to, an electrical connection having one or more wires, a portable computer disk, a hard disk, random access memory (RAM), read-only memory (ROM), erasable programmable read-only memory (EPROM or flash memory), optical fiber, a portable compact disk read-only memory (CD-ROM), an optical storage device, a magnetic storage device, or any suitable combination thereof. In the embodiment, the computer-readable storage medium may be any tangible medium that contains or stores a program that can be used by or in conjunction with an instruction execution system, system, or device. The program code included on the computer-readable storage medium may be transmitted using any suitable medium, including but not limited to wires, optical cables, radio frequency (RF), etc., or any suitable combination thereof.

The computer-readable storage medium may be included in the electronic device, or may exist independently without being incorporated into the electronic device.

The above-mentioned computer-readable storage medium carries one or more programs. When the above-mentioned one or more programs are executed by an electronic device, the electronic device: synchronously collects physiological signals, the physiological signals include heart sound signals; obtains a segmentation threshold corresponding to the heart sound signal, segments the heart sound signal based on the segmentation threshold to obtain a first heart sound and a second heart sound; and determines the systolic duration and the diastolic duration based on the first heart sound and the second heart sound.

Computer program code for performing operations of the present application may be written in one or more programming languages, or a combination thereof, including object-oriented programming languages such as Java, Smalltalk, and C++, as well as conventional procedural programming languages such as “C” or similar programming languages. The program code may execute entirely on the user's computer, partially on the user's computer, as a stand-alone software package, partially on the user's computer and partially on a remote computer, or entirely on the remote computer or server. In the case of a remote computer, the remote computer may be connected to the user's computer via any type of network, including a local area network (LAN) or a wide area network (WAN), or may be connected to an external computer (e.g., via the Internet using an Internet service provider).

The flow charts and block diagrams in the accompanying drawings illustrate the possible implementation architecture, functions and operations of the system, method and computer program product according to various embodiments of the present application. In this regard, each box in the flow chart or block diagram can represent a module, program segment, or a part of code, and the module, program segment, or a part of code contains one or more executable instructions for realizing the specified logical function. It should also be noted that in some alternative implementations, the functions marked in the box can also occur in a different order than that marked in the accompanying drawings. For example, two boxes represented in succession can actually be executed substantially in parallel, and they can sometimes be executed in the opposite order, depending on the functions involved. It should also be noted that each box in the block diagram and/or flow chart, and the combination of the boxes in the block diagram and/or flow chart, can be implemented with a dedicated hardware-based system that performs the specified function or operation, or can be implemented with a combination of dedicated hardware and computer instructions.

The modules involved in the embodiments described in the present application may be implemented in software or hardware, the name of a module does not necessarily limit the unit itself.

The computer-readable storage medium provided herein stores computer-readable program instructions for executing the aforementioned heart sound segmentation method, thereby solving the technical problem of reducing resource consumption during heart sound segmentation. Compared to the related art, the computer-readable storage medium provided in the embodiment of the present application achieves the same beneficial effects as the heart sound segmentation methods provided in the first, second, or third embodiments above, and will not be further elaborated here.

### Seventh Embodiment

An embodiment of the present application further provides a computer program product, including a computer program, which implements the steps of the above-mentioned heart sound segmentation method when executed by a processor.

The computer program product provided in the present application can solve the technical problem of reducing resource consumption in heart sound segmentation. Compared to related arts, the computer program product provided in the embodiment of the present application offers the same beneficial effects as the heart sound segmentation methods provided in the first, second, or third embodiments above, and will not be further elaborated here.

The above are only some embodiments of the present application and do not limit the patent scope of the present application. Any equivalent structure or equivalent process transformation made using the contents of the present application specification and drawings, or directly or indirectly applied in other related arts, are also included in the patent processing scope of the present application.

What is claimed is:

1. A heart sound segmentation method, comprising:
synchronously collecting physiological signals, wherein the physiological signals comprise heart sound signals;
obtaining a segmentation threshold corresponding to the heart sound signal, and segmenting the heart sound signal based on the segmentation threshold to obtain a first heart sound and a second heart sound; and
determining a systolic duration and a diastolic duration of the heart sound signal according to the first heart sound and the second heart sound.

2. The heart sound segmentation method according to claim 1, wherein the physiological signal further comprises an electrocardiogram signal and a pulse signal, and before the obtaining the segmentation threshold corresponding to the heart sound signal, the method further comprises:
determining a QRS wave peak point of the electrocardiogram signal, and using a time corresponding to the QRS wave peak point as a first time;
determining a trough point of the pulse signal, and using a time corresponding to the trough point as a second time;
determining a first zero crossing point and a second zero crossing point of the pulse signal, using a time corresponding to the first zero crossing point as a third time, and using a time corresponding to the second zero crossing point as a fourth time;
determining a first threshold corresponding to the first time and the second time, and determining a second threshold corresponding to the third time and the fourth time; and
using the first threshold and the second threshold as segmentation thresholds.

3. The heart sound segmentation method according to claim 2, wherein the determining the first threshold corresponding to the first time and the second time comprises:
obtaining or determining a heart sound envelope signal corresponding to the heart sound signal;
using a heart sound peak value of the heart sound envelope signal between the first time and the second time as a first heart sound peak value; and
adjusting the first heart sound peak value based on a preset adjustment coefficient to obtain the first threshold, wherein the first threshold is less than the first heart sound peak value.

4. The heart sound segmentation method according to claim 2, wherein the determining the second threshold corresponding to the third time and the fourth time comprises:
obtaining or determining a heart sound envelope signal corresponding to the heart sound signal;
using a heart sound peak value of the heart sound envelope signal between the third time and the fourth time as a second heart sound peak value; and
adjusting the second heart sound peak value based on a preset adjustment coefficient to obtain the second threshold, wherein the second threshold is less than the second heart sound peak value.

5. The heart sound segmentation method according to claim 1, wherein the segmentation threshold comprises a first threshold and a second threshold, and the segmenting the heart sound signal based on the segmentation threshold comprises:
obtaining or determining a heart sound envelope signal corresponding to the heart sound signal;
using a duration window in which a signal value of the heart sound envelope signal is greater than the first threshold as a first duration window;
using a duration window in which the signal value of the heart sound envelope signal is greater than the second threshold as a second duration window; and
using the heart sound signal within the first duration window as the first heart sound, and using the heart sound signal within the second duration window as the second heart sound.

6. The heart sound segmentation method according to claim 1, wherein the determining the systolic duration and the diastolic duration of the heart sound signal according to the first heart sound and the second heart sound comprises:
determining cardiac cycles comprised in the heart sound signal, and traversing each of the cardiac cycles sequentially;
using an end time of the first heart sound in the traversed cardiac cycle as a first end time, determining a next cardiac cycle corresponding to the traversed cardiac cycle, and using a start time of the first heart sound in the next cardiac cycle as a first start time;
using a start time of the second heart sound in the traversed cardiac cycle as a second start time, and using an end time of the second heart sound in the traversed cardiac cycle as a second end time;
using a time difference between the first end time and the second start time as the systolic duration;
using a time difference between the first start time and the second end time as the diastolic duration;
after each cardiac cycle is traversed, determining an average systolic duration of all the systolic durations and an average diastolic duration of all the diastolic durations; and
using the average systolic duration as the systolic duration of the heart sound signal, and using the average diastolic duration as the diastolic duration of the heart sound signal.

7. A heart sound segmentation apparatus, comprising:
a signal acquisition module, configured to synchronously collect physiological signals, wherein the physiological signals comprise heart sound signals;
a segmentation module, configured to obtain a segmentation threshold corresponding to the heart sound signal, and segment the heart sound signal based on the segmentation threshold to obtain a first heart sound and a second heart sound;
a determination module, configured to determine a systolic duration and a diastolic duration of the heart sound signal according to the first heart sound and the second heart sound.

8. An electronic device, comprising:
at least one processor; and
a memory communicatively connected to the at least one processor,
wherein the memory stores instructions that can be executed by the at least one processor, and the instructions are executed by the at least one processor to enable the at least one processor to perform the heart sound segmentation method according to claim 1.

9. A non-transitory computer-readable storage medium, characterized in that the readable storage medium is a computer-readable storage medium, and a program for implementing a heart sound segmentation method is stored on the computer-readable storage medium, and the program for implementing the heart sound segmentation method is executed by a processor to implement the heart sound segmentation method according to claim 1.
